(19) United States
(12) Patent Application Publication
(10) Pub. No.: US 2004/0259850 A1
(43) Pub. Date: Dec. 23, 2004

(54) Method for treating or preventing symptoms of hormonal variation including hot flashes

(76) Inventors: Stephen E. Alves, New York, NY (US); Milton I Hammond, Somerville, NJ (US); Samuel D Wright, Westfield, NJ (US)

Correspondence Address: MERCK AND CO INC, P O BOX 2000, RAHWAY, NJ, 070650907

(21) Appl. No.: 10/494,548

(22) PCT Filed: Oct. 25, 2002
(86) PCT No.: PCT/US02/34531

Related U.S. Application Data
(60) Provisional application No. 60/334,856, filed on Oct. 31, 2001.

(51) Int. Cl. A61K 31/56

(52) U.S. Cl. 514/169

(57) ABSTRACT

A tachykinin receptor antagonist, in particular a neurokinin-1 receptor antagonist, is useful for the treatment or prevention of hot flashes associated with hormonal variation in a patient.

### BACKGROUND OF THE INVENTION

Hot flashes or “night sweats”, manifested as an increase in skin temperature, occur commonly in menopausal women. This symptom is characteristic of a heat-dissipation response that consists of the sudden onset of sweating on the face, neck and chest, as well as peripheral withdrawal vasodilation. (Freedman, R. R. “Physiology of Hot Flashes,” Amer. J. Human Biol., 13 (4): 453-464 (2001)). Such an episode generally lasts several minutes and is evidenced by a visible flushing of the skin. Often dizziness, palpitations and diaphoresis accompany such episodes. Such symptoms can disrupt sleep and interfere with the quality of life. Although the cause of hot flashes are not completely understood, they are associated with the periods of hormonal variation women experience during menopause where estrogen levels decline. Thus, it is not surprising that hot flashes also occur in a high percentage of women undergoing breast cancer treatment by using the anti-estrogen drug tamoxifen. It is thought that a disorder of thermoregulation exists resulting from a transient lowering of the hypothalamic temperature regulatory set point (Kronenberg et al., “Thermoregulatory Physiology of Menopausal Hot Flashes: A Review,” Can. J. Physiol. Pharmacol., 65:1312-1324 (1987)).

Hot flashes (flushes) and sweating secondary to vasomotor instability affect 75% of women during menopause. Most have hot flushes for >1 yr, and 25 to 50% for >5 yr. The woman feels warm or hot and may perspire, sometimes profusely. The skin, especially of the head and neck, becomes red and warm. The flush, which may last from 30 sec to 5 min, may be followed by chills. Vasomotor symptoms of the hot flash coincide with the onset of lutenizing hormone pulses, but not every increase in lutenizing hormone is associated with a hot flash, suggesting that hypothalamic control of lutenizing hormone pulses is independent of that of flushes. This independence is confirmed by the occurrence of hot flashes in women who have had pituitary failure and do not secrete lutenizing hormone and folicular stimulating hormone. Men who are undergoing androgen-deprivation therapy may also experience hot flashes following a bilateral orchiectomy or treatment with a gonadotrophin-releasing-hormone agonist for metastatic prostate cancer.

Currently, the established treatment for hot flashes in patients is either hormone replacement therapy (HRT-estrogen and progesterone) or estrogen-replacement therapy (ERT). However such treatments are inappropriate for patients previously diagnosed with breast cancer, since either estrogen or progesterone may be associated with an increased risk of cancer recurrence. Furthermore, women with a history of clotting or severe migraines are averse to undergoing such therapy because other estrogen-mediated side effects (uterine cancer, vaginal bleeding, and vein thrombosis) may emerge.

Other than estrogen-replacement therapy, there are no effective means to alleviate hot flashes. Low dose oral megestrol acetate, a progestational agent, was shown to reduce the frequency of hot flashes in both men and women in a short term study (Loprinzi et al., “Megestrol Acetate for the Prevention of Hot Flashes,” N. Engl. J. Med. 331:347-351 (1994)). However, chronic adrenal insufficiency can be a side effect of low dose megestrol acetate when taken long term. Consequently, novel drugs that do not interfere with hormonal levels yet treat vasomotor symptoms effectively are needed as alternative treatment for cancer patients.

In efforts to discover non-hormonal treatment for hot flashes, researchers conducted studies which indicated that transdermal clonidine, a centrally active α-agonist, had only a moderate effect on the frequency and severity of hot flashes in tamoxifen-treated women (Goldberg et al., “Transdermal Clonidine for Ameliorating Tamoxifen-induced Hot Flashes,” J. Clin. Onc. 12:155-158 (1994)). Similar limited results were obtained when given to men for relief of post-orchiectomy hot flashes (Loprinzi, et al., “Transdermal Clonidine for Amerliorating Post-Orchiectormy Hot Flashes,” J. Urol. 151: 634-636 (1994)). Clonidine only mildly ameliorates hot flashes while estrogen can virtually eliminate them. Another mode of treating or preventing vasomotor symptoms is by influencing the serotonin levels in the body. Mirtazapine appears to have an ameliorating effect on hot flushes and perspiration bouts. It is postulated that the 5-HT (2A) blocking properties of mirtazapine affect the serotonergic system (Waldinger et al., “Treatment of Hot Flashes with Mirtazapine: Four Case Reports,” Maturitas 36: 165-168 (2000)). Early studies suggest that compounds such as venlafaxine can serve as an effective non-hormonal method to diminish hot flashes. (Loprinzi et al., “Venlafaxine in Management of Hot Flashes in Survivors of Breast Cancer: a Randomized Controlled Trial,” Lancet 356: 2059-2063 (2000)). However, there are side effects associated with venlafaxine such as mouth dryness, decreased appetite, nausea and constipation.

Accordingly, there is a need for an alternative method of treating symptoms of hormonal variation, including hot flashes, which overcomes the deficiencies in the art.

The neuropeptide receptors for substance P (neurokinin-1; NK-1) are widely distributed throughout the mammalian nervous system (especially brain and spinal ganglia), the circulatory system and peripheral tissues (especially the duodenum and jejunum) and are involved in regulating a number of diverse biological processes. This includes sensory perception of olfaction, vision, audition and pain, movement control, gastric motility, vasodilation, salivation, and micturition (B. Pernow, Pharmacol. Rev., 1983, 35, 85-141). Substance P (SP) is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to Substance P, the known mammalian tachykinins include neurokinin A and neurokinin B. The current nomenclature designates the receptors for substance P, neurokinin A, and neurokinin B as neurokinin-1, neurokinin-2, and neurokinin-3, respectively. Substance P mediates its biological actions mainly through selectively binding to the neurokinin-1 receptor (von Euler, U.S.; Gaddum J. H.; J. Physiol. (London) 72, 74-87 (1931)). Substance P/neurokinin-1 receptor antagonists have potential in the treatment of rheumatoid arthritis, pain, migraine, inflammation and in the prevention of emetic episodes. Neurokinin-1 (NK-1; substance P) receptor antagonists are being developed for the treatment of a number of physiological disorders associated with an excess or imbalance of tachykinins, and in particular substance P. Examples of such conditions include disorders of the central nervous system such as anxiety, depression and psychosis. Other conditions or diseases in which substance P has been implicated include disorders of the respiratory system (asthma), inflammatory diseases (rheumatoid arthritis), and gastrointestinal disorders (ulcerative colitis and Crohn's disease).

Prior to the present invention, however, it has not been disclosed or suggested that a neurokinin-1 receptor antagonist would be useful for the treatment of hormonal variation. Currently there are only limited means for treating or preventing hot flashes and other vasomotor symptoms. It would be desirable to have a therapeutic agent that prevents hot flashes without the risks and side effects associated with existing agents such as estrogen. In view of the shortcomings of existing agents, there is a need for new effective methods for treating or preventing hot flashes.

### SUMMARY OF THE INVENTION

The present invention is directed to a method for treating or preventing hormonal variation or symptoms of hormonal variation in a patient comprising the administration of a tachykinin receptor antagonist, in particular a neurokinin-1 receptor antagonist in a therapeutically effective amount. The present invention is further directed to a method for treating or preventing vasomotor symptoms in a patient comprising the administration of a tachykinin receptor antagonist, in particular a neurokinin-1 receptor antagonist, in a therapeutically effective amount. The present invention further relates to the use of a tachykinin receptor antagonist, in particular a neurokinin-1 receptor antagonist, for the treatment, prevention or amelioration of hot flashes or vasomotor symptoms in a patient such as a woman comprising the administration of a tachykinin receptor antagonist, in particular a neurokinin-1 receptor antagonist.

### DESCRIPTION OF THE INVENTION

The present invention is directed to a method for treating or preventing hormonal variation or symptoms of hormonal variation in a patient comprising the administration of a tachykinin receptor antagonist, in particular a neurokinin-1 receptor antagonist in a therapeutically effective amount.

The present invention is further directed to a method for treating or preventing vasomotor symptoms in a patient comprising the administration of a tachykinin receptor antagonist, in particular a neurokinin-1 receptor antagonist, in a therapeutically effective amount.

The present invention is also directed to a method for treating or preventing vasomotor symptoms associated with hormonal variation in a patient comprising the administration of a tachykinin receptor antagonist, in particular a neurokinin-1 receptor antagonist, in a therapeutically effective amount.

The present invention is further directed to a method for alleviating, managing or ameliorating the symptoms attendant to menopause in a female patient comprising the administration of a tachykinin receptor antagonist, in particular a neurokinin-1 receptor antagonist, in a therapeutically effective amount.

The present invention further relates to a method of treating hot flashes in a patient, which is carried out by providing a compound, which binds to the neurokinin-1 receptor, and administering the compound to a patient experiencing hot flashes under condition effective to treat hot flashes.

The present invention is further directed to a method for treating, preventing or ameliorating hot flashes or night sweats in a patient comprising the administration of a tachykinin receptor antagonist, in particular a neurokinin-1 receptor antagonist, to the patient in a therapeutically effective amount.

In a preferred embodiment, the present invention provides a method for treating or preventing menopausal hot flashes in a female patient comprising the administration of a tachykinin receptor antagonist, in particular a neurokinin-1 receptor antagonist, in a therapeutically effective amount. The present invention is of great benefit to women who experience hot flashes during the onset of their menopausal period, because the tachykinin receptor antagonist acts to alleviate and/or prevent such adverse symptoms.

In a preferred embodiment, the present invention further provides a method for ameliorating the symptoms attendant to hormonal variation in a male patient undergoing hormonal therapy post-orchiectomy comprising the administration of a tachykinin receptor antagonist, in particular a neurokinin-1 receptor antagonist, in a therapeutically effective amount.

The present invention is further directed to a method for ameliorating hot flashes comprising the administration of a tachykinin receptor antagonist, in particular a neurokinin-1 receptor antagonist, in combination with one or more active agents selected from the group consisting of estrogen and androgen receptor modulators and peptide hormones.

The present invention is useful in any mammal suffering from hormonal variation, but a preferred patient is a human. Although the present invention is applicable to both women and men, a more preferred subject is a woman. An even more preferred subject is an elderly woman, or a woman who is menopausal woman or otherwise suffering from hormonal variations or symptoms of hormonal changes. The present invention may be employed in a patient where hormonal variations are naturally induced (such as by menopause, including perimenopause, climacteric menopause or premature menopause), drug-induced (such as by anti-estrogen or anti-androgen therapy) or surgically induced (such as by hysterectomy, oopherectomy, orchiectomy, chemotherapy, radiation of the pelvis, or any process that impairs ovarian blood supply).

The tachykinin receptor antagonists of use in the present invention may be any tachykinin receptor antagonists known from the art. Preferably, the tachykinin receptor antagonist is a neurokinin-1 (NK-1) or neurokinin-2 (NK-2) receptor antagonist, especially a neurokinin-1 (NK-1) receptor antagonist.

Neurokinin-1 receptor antagonists are herein defined as chemical compounds capable of binding to the neurokinin-1 receptor sites in mammalian tissue, and blocking the actions of neurokinin-1 or substance P in one or more tissues.

The neurokinin-1 receptor antagonist may be peptidal or non-peptidal in nature; however, the use of a non-peptidal neurokinin-1 receptor antagonist is preferred. In a preferred embodiment, the neurokinin-1 receptor antagonist is a CNS-penetrant neurokinin-1 receptor antagonist. In addition, for convenience the use of an orally active neurokinin-1 receptor antagonist is preferred. To facilitate dosing, it is also preferred that the neurokinin-1 receptor antagonist is a long acting neurokinin-1 receptor antagonist. Particularly preferred classes of neurokinin-1 receptor antagonists of use in the present invention are those compounds that are orally active and long acting.

Neurokinin-1 (NK-1, Substance P) receptor antagonists are under development for the treatment of a number of physiological disorders associated with an excess or imbalance of tachykinins, and in particular substance P. Neurokinin-1 receptor antagonists of use in the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,889,042, 5,962,505, 6,011,006, 6,107,331, 6,245,812; European Patent Publication Nos. EP 0 309 473, 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632, 0 776 893, and 0 943 329; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, 97/49710, 00/12087, 98/34608, 99/03880, 99/44627 and 00/63228, 00/07598, 01/07037; and in British Patent Publication Nos. 2 266 529, 2 268 931, 2 269 170, 2 269 590, 2 271 774, 2 292 144, 2 293 168, 2 293 169, and 2 302 689.

Specific neurokinin-1 receptor antagonists of use in the present invention include:

(±)-(2R3R,2S3S)—N-{[2-cyclopropoxy-5-(trifluoromethoxy)-phenyl]methyl}-2-phenylpiperidin-3-amine;

2-(S)-(3,5-bis(trifluoromethyl)benzyloxy)-3(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine;

2-(S)-(3,5-bis(trifluoromethyl)benzyloxy)-4-(3-(5-oxo-1H,4H-1,2,4-triazolo)methyl)-3-(S)-phenyl-morpholine;

2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(5-(N,N-dimethylamino)methyl-1,2,3-triazol-4-yl)methyl-3-(S)-phenylmorpholine;

2-(R)-(1-(R)-(3,5-bis(trifluoromethyl)phenyl)ethoxy)-4-(5-(N,N-dimethylamino)methyl-1,2,3-triazol-4-yl)methyl-3-(S)-(4-fluorophenyl)morpholine;

(3S,5R,6S)-3-[2-cyclopropoxy-5-(trifluoromethoxy)phenyl]-6-phenyl-1-oxa-7-aza-spiro[4.5]decane;

(3R,5R,6S)-3-[2-cyclopropoxy-5-(trifluoromethoxy)phenyl]-6-phenyl-1-oxa-7-aza-spiro[4.5]decane;

2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;

2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;

2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;

2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;

or a pharmaceutically acceptable salt thereof.

The preparation of such compounds is fully described in the aforementioned patents and publications.

Suitable pharmaceutically acceptable salts of the compounds of use in the present invention include acid addition salts which may be formed by mixing a solution of the compound with a solution of a pharmaceutically acceptable non-toxic acid such as hydrochloric acid, fumaric acid, maleic acid, succinic acid, acetic acid, citric acid, tartaric acid, carbonic acid, phosphoric acid or sulfuric acid. Salts of amine groups may also comprise the quaternary ammonium salts in which the amino nitrogen atom carries an alkyl, alkenyl, alkynyl or aralkyl group. Where the compound carries an acidic group, such as a carboxylic acid group, the present invention also contemplates salts thereof, preferably non-toxic pharmaceutically acceptable salts thereof, such as the sodium, potassium and calcium salts thereof.

The above compounds are only illustrative of the neurokinin-1 (NK-1) receptor antagonists that are currently under investigation. As this listing of compounds is not meant to be comprehensive, the methods of the present invention may employ any neurokinin-1 receptor antagonist, in particular a neurokinin-1 receptor antagonist that is orally active and long acting. Accordingly, the present invention is not strictly limited to any particular structural class of compound.

The present invention accordingly provides the use of a neurokinin-1 receptor antagonist for the manufacture of a medicament for treating or preventing vasomotor symptoms or ameliorating the symptoms attendant to hormonal variation induced naturally, by a drug or by surgery.

In a further aspect of the present invention, there is provided a pharmaceutical composition for treating or preventing vasomotor symptoms in a patient comprising a neurokinin-1 receptor antagonist, together with at least one pharmaceutically acceptable carrier or excipient.

The identification of a compound as a tachykinin receptor antagonist, in particular, a neurokinin-1 receptor antagonist, and thus able to have utility in the present invention may be readily determined without undue experimentation by methodology well known in the art.

The term “hot flashes”, as used herein means symptoms such as vasomotor symptoms (i.e. regarding the size of blood vessels) that may include sweating on the face, neck and chest and typically manifest during periods of low levels of estrogen in the patient's body. Hormonal changes may be naturally induced (menopausal period), drug-induced (anti-estrogen or anti-androgen therapy) or surgically induced (oopherectomy, orchiectomy).

The term “effective amount”/“therapeutically effective amount”, as used herein, means that amount of the neurokinin-1 receptor antagonist and (where present) the additional active agent(s), that will elicit the desired therapeutic effect or response when administered in accordance with the prescribed treatment regimen. A preferred therapeutically effective amount of the neurokinin-1 receptor antagonist and where present the additional active agent(s) is an amount that for the patient treats or prevents hormonal variation or symptoms of hormonal variation such as vasomotor symptoms for example hot flashes.

Although the present invention is not limited to a specific mechanism of action, the inventors postulate that a tachykinin receptor antagonist, in particular a neurokinin-1 (substance P) receptor antagonist would be effective in the treatment of vasomotor symptoms associated with hormonal variation. In accordance with the present invention, administration of a neurokinin-1 receptor antagonist in therapeutic amounts can alleviate the effects of excessive or imbalanced amounts of substance P.

A tachykinin receptor antagonist may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

As noted above, the tachykinin receptor antagonist/neurokinin-1 receptor antagonist may be formulated in a single pharmaceutical composition or alternatively in individual pharmaceutical compositions for simultaneous, separate or sequential use in accordance with the present invention. Preferably the compositions according to the present invention are in unit dosage forms such as tablets, pills, capsules, powders, granules, solutions or suspensions, or suppositories, for oral, parenteral or rectal administration, by inhalation or insufflation or administration by transdermal patches or by buccal cavity absorption wafers. Oral dosage forms are particularly preferred (e.g. tablets, capsules, pills or wafers). For preparing solid compositions such as tablets, the principal active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g. elixirs and syrups, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Preferably the compositions according to the present invention are in unit dosage forms such as tablets, pills, capsules, powders, granules, solutions or suspensions, or suppositories, for oral, parenteral or rectal administration, by inhalation or insufflation or administration by transdermal patches or by buccal cavity absorption wafers. For preparing solid compositions such as tablets, the principal active ingredient is mixed with a pharmaceutical carrier, e.g.conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil, peanut oil or soybean oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

Preferred compositions for administration by injection include those comprising a neurokinin-1 receptor antagonist as the active ingredient, in association with a surface-active agent (or wetting agent or surfactant) or in the form of an emulsion (as a water-in-oil or oil-in-water emulsion).

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and preferably between 0.1 and 2.5%. It will be appreciated that other ingredients may be added, for example mannitol or other pharmaceutically acceptable vehicles, if necessary. Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion will preferably comprise fat droplets between 0.1 and 1.0 μm, particularly 0.1 and 0.5 μm, and have a pH in the range of 5.5 to 8.0.

Compositions for inhalation or insufflation include solutions and suspensions in pharmaceutically acceptable, aqueous or organic solvents or mixtures thereof, and powder. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulised by use of inert gases. Nebulised solutions may be breathed directly from the nebulising device or the nebulising device may be attached to a facemask, tent or intermittent positive pressure breathing machine. Solution, suspension or powder compositions may be administered, preferably orally or nasally, from devices that deliver the formulation in an appropriate manner.

Compositions of the present invention may also be presented for administration in the form of transdermal patches using conventional technology. The compositions may also be administered via the buccal cavity using, for example, absorption wafers. Compositions in the form of tablets, pills, capsules or wafers for oral administration are particularly preferred.

It will be known to those skilled in the art that there may be numerous compounds which may be used for treating or preventing vasomotor symptoms arising from hormonal variation in a patient. Combinations of these therapeutic agents, some of which have also been mentioned herein with a tachykinin receptor antagonist, will bring additional, complementary, and often synergistic properties to enhance the desirable properties of these various therapeutic agents. In these combinations, the tachykinin receptor antagonist and the therapeutic agents may be independently present in dose ranges from one one-hundredth to one times the dose levels which are effective when these compounds are used singly. In such combination therapy, the tachykinin receptor antagonist may be administered with the other therapeutic agent (e.g. concurrently, concombinantly, sequentially, or in a unitary formulation) such that their therapeutic efficacy overlap.

The tachykinin receptor antagonist may be administered in combination with estrogens, estrogen receptor modulators, estrogen agonists, androgen receptor modulators, peptide hormones, sedatives, hypnotics, anxiolytics, antipsychotics, antianxiety agents, minor tranquilizers, benzodiazepines, barbituates, serotonin (5-HT) agonists, selective serotonin reuptake inhibitors (SSRI's), 5HT-2 antagonists, non-steroidal anti-inflammatory drugs, oral contraceptives, progesterone, progestin, monoamine oxidase inhibitors, carbohydrate mixtures and the like, or the tachykinin receptor antagonist may be administered in conjunction with the use of physical methods such as cooling agents.

For example, for treating or preventing symptoms arising from hormonal variation in a patient, a tachykinin receptor antagonist may be given in combination with such compounds as: estrogen, progesterone, clonidine, venlafaxine, megestrol acetate, mirtazapine, a non-steroidal antiinflammatory, such as acetomeniphen, alprostadil, asprin, diclofenac, etodolac, ibuprofen, indomethacin, ketoprofe, ketorolac tromethamine, misoprostol, nabumetone, naproxen, naproxen sodium, oxaprozin, piroxicam, spironolactone, spironolactone with hydrochlorothiazide, or trovafloxacin; a corticosteroid; a selective cyclooxygenase-2 inhibitor, such as celecoxib, etoricoxib, parecoxib, rofecoxib, valdecoxib, meloxicam, flosulide, nimesulide, MK-663, NS 398, DuP 697, SC-58125, SC-58635, or RS 57067; adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clometherone, clomipramine, cloperidone, clorazepate, clorethate, clozapine, cyprazepam, delmadinone, desipramine, dexclamol, diazepam, dichloralphenazone, divalproex, diphenhydramine, doxepin, droloxifene, estazolam, estradiol, estrogen, ethchlorvynol, etomidate, fenobam, flunitrazepam, flurazepam, fluvoxamine, fluoxetine, fosazepam, glutethimide, halazepam, hydroxyzine, idoxifene, imipramine, lithium, leucine, leuprolide, lorazepam, lormetazepam, maprotiline, mecloqualone, melatonin, mephobarbital, meprobamate, methaqualone, midaflur, midazolam, nafoxidine, nefazodone, nitromifene, nisobamate, nitrazepam, nociceptin, nortriptyline, ormeloxifene, oxazepam, paraldehyde, paroxetine, pentobarbital, perlapine, perphenazine, phenelzine, phenobarbital, prazepam, progesterone, promethazine, propofol, protriptyline, quazepam, raloxifene, reclazepam, roletamide, secobarbital, sertraline, suproclone, tamoxifene, temazepam, thioridazine, toremifene, tracazolate, tranylcypromaine, trazodone, trioxifene, triazolam, trepipam, tricetamide, triclofos, trifluoperazine, trimetozine, trimipramine, uldazepam, valproate, venlafaxine, zaleplon, zolazepam, zolpidem, and salts thereof, and combinations thereof, and the like, as well as admixtures and combinations thereof.

Typically, the individual daily dosages for these combinations may range from about one-fifth of the minimally recommended clinical dosages to the maximum recommended levels for the entities when they are given singly.

To illustrate these combinations, a tachykinin receptor antagonist effective clinically at a given daily dose range may be effectively combined, at levels which are equal or less than the daily dose range, with the aforementioned compounds. It will be readily apparent to one skilled in the art that the tachykinin receptor antagonist may be employed with other agents for treating or preventing vasomotor symptoms including hot flashes in a patient.

Naturally, these dose ranges may be adjusted on a unit basis as necessary to permit divided daily dosage and, as noted above, the dose will vary depending on the nature and severity of the disease, weight of patient, special diets and other factors. These combinations may be formulated into pharmaceutical compositions as known in the art and as discussed herein.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease or disorder, the patient's weight, special diets then being followed by a patient, concurrent medication, the intrinsic tachykinin receptor antagonist activity of the compound, the bioavailability upon oral administration of the compound and other factors which those skilled in the art will recognize.

In the treatment of a condition in accordance with the present invention, an appropriate dosage level will generally be about 0.01 μg to 50 mg per kg patient body weight per day which may be administered in single or multiple doses. Preferably, the dosage level will be about 0.1 μg to about 25 mg/kg per day; more preferably about 0.5 μg to about 10 mg/kg per day. For example, for treating or preventing vasomotor symptoms including hot flashes in a patient, a suitable dosage level is about 0.1 μg to 25 mg/kg per day, preferably about 0.5 μg to 10 mg/kg per day, and especially about 1 μg to 5 mg/kg per day. In larger mammals, for example humans, a typical indicated dose is about 300 μg to 400 mg orally. A compound may be administered on a regimen of several times per day, for example 1 to 4 times per day, preferably once or twice per day. When using an injectable formulation, a suitable dosage level is about 0.1 μg to 10 mg/kg per day, preferably about 0.5 μg to 5 mg/kg per day, and especially about 1 μg to 1 mg/kg per day. In larger mammals, for example humans, a typical indicated dose is about 100 μg to 100 mg i.v. A compound may be administered on a regimen of several times per day, for example 1 to 4 times per day, preferably once or twice per day, and more preferably once a day.

Pharmaceutical compositions of the present invention may be provided in a solid dosage formulation preferably comprising about 100 μg to 500 mg active ingredient, more preferably comprising about 100 μg to 250 mg active ingredient. The pharmaceutical composition is preferably provided in a solid dosage formulation comprising about 100 μg, 1 mg, 5 mg, 10 mg, 25 mg, 50 mg, 100 mg, 200 mg or 300 mg active ingredient. A minimum dosage level for the neurokinin-1 receptor antagonist is generally about 5 mg per day, preferably about 10 mg per day and especially about 20 mg per day. A maximum dosage level for the neurokinin-1 receptor antagonist is generally about 1500 mg per day, preferably about 1000 mg per day and especially about 500 mg per day.

It will be appreciated that the amount of the neurokinin-1 receptor antagonist required for use in treating or preventing hot flashes or ameliorating the symptoms attendant to hormonal variation in a patient will vary not only with the particular compounds or compositions selected but also with the route of administration, the nature of the condition being treated, and the age and condition of the patient, and will ultimately be at the discretion of the patient's physician or pharmacist. The length of time during which a tachykinin receptor antagonist will be given varies on an individual basis.

Particularly preferred neurokinin-1 receptor antagonists for use in the present invention are compounds that are potent neurokinin-1 receptor antagonists, i.e. compounds with a neurokinin-1 receptor affinity (IC₅₀) of less than 10 nM.

A particularly preferred class of neurokinin-1 receptor antagonist of use in the present invention are those compounds which are orally active and long acting. The use of this sub-class of neurokinin-1 receptor antagonists for treating or preventing hormonal variation, including vasomotor symptoms such as hot flashes in a patient represents a further aspect of the present invention.

Thus, the present invention provides the use of a neurokinin-1 receptor antagonist in an oral, once-a-day medicament for treating or preventing hormonal variation in a patient. The compounds of this class exhibit advantageous benefits when compared against conventional methods for treating or preventing hot flashes in a patient.

In particular, the present invention provides a means for the identification of neurokinin-1 receptor antagonists that would be especially effective in an oral once-a-day medicament for treating or preventing vasomotor symptoms in a patient.

Furthermore, the exceptional pharmacology of the class of neurokinin-1 receptor antagonists of use in the present invention results in a rapid onset of action.

The present invention accordingly provides the use of an orally active, long acting neurokinin-1 receptor antagonist (as hereinafter defined) for the manufacture of a medicament adapted for oral administration for treating or preventing vasomotor symptoms arising from a hormonal variation in a patient.

The present invention also provides a method for treating or preventing hot flashes in a patient, which method comprises the oral administration to a patient in need of such treatment of an effective amount of an orally active, long acting neurokinin-1 receptor antagonist (as defined herein).

In a further aspect of the present invention, there is provided an oral pharmaceutical composition for treating or preventing hot flashes in a patient which comprises an orally active, long acting neurokinin-1 receptor antagonist (as hereinafter defined), together with a pharmaceutically acceptable carrier or excipient.

It will be appreciated to those skilled in the art that reference herein to treatment extends to prophylaxis (prevention) as well as the treatment of the noted diseases/disorders and symptoms.

The following examples are provided for the purpose of further illustration only and are not intended to be limitations on the disclosed invention.

### EXAMPLE 1

NK-1 Receptor Binding Assay

NK-1 receptor binding assays are performed in intact Chinese hamster ovary (CHO) cells expressing the human neurokinin-1 receptor using a modification of the assay conditions described by Cascieri et al, J. Pharmacol. Exp. Ther., 1992, 42, 458. Typically, the receptor is expressed at a level of 3×10⁵ receptors per cell. Cells are grown in monolayer culture, detached from the plate with enzyme-free dissociation solution (Speciality Media Inc.), and washed prior to use in the assay. ¹²⁵I-Tyr⁸-substance P (0.1 nM, 2000 Ci/mmol; New England Nuclear) is incubated in the presence or absence of test compounds (dissolved in 5 μl dimethylsulphoxide, DMSO) with 5×10⁴ CHO cells. Ligand binding is performed in 0.25 ml of 50 nM Tris-HCl, pH7.5, containing 5 mM MnCl₂, 150 mM NaCl, 0.02% bovine serum albumin (Sigma), 50 μg/ml chymostatin (Peninsula), 0.1 nM phenylmethylsulphonyl fluoride, 2 μg/ml pepstatin, 2 μg/ml leupeptin and 2.8 μg/ml furoyl saccharine. The incubation proceeds at room temperature until equilibrium is achieved (>40 minutes) and the receptor-ligand complex is harvested by filtration over GF/C filters pre-soaked in 0.1% polyethylenimine using a Tomtek 96-well harvester. Non-specific binding is determined using excess substance P (1 μM and represents <10% of total binding.

Particularly preferred neurokinin-1 receptor antagonists of use in the present invention are compounds which are potent neurokinin-1 receptor antagonists, i.e. compounds with a neurokinin-1 receptor affinity (IC₅₀) of less than 10 nM, favorably less than 2 nM and preferably less than 1 nM.

The following examples illustrate pharmaceutical compositions according to the invention.

### EXAMPLE 2

Tablet formulation containing 50-300 mg of NK-1
Receptor Antagonist

| | Amount (mg) | | |
|---|---|---|---|
| NK-1 receptor antagonist | 50.0 | 100.0 | 300.0 |
| Microcrystalline cellulose | 80.0 | 80.0 | 80.0 |
| Modified food corn starch | 80.0 | 80.0 | 80.0 |
| Lactose | 189.5 | 139.5 | 439.5 |
| Magnesium Stearate | 0.5 | 0.5 | 0.5 |

The active ingredient, cellulose, lactose and a portion of the cornstarch are mixed and granulated with 10% cornstarch paste. The resulting granulation is sieved, dried and blended with the remainder of the cornstarch and the magnesium stearate. The resulting granulation is then compressed into tablets containing 50 mg, 100 mg and 300 mg of the NK-1 receptor antagonist per tablet.

### EXAMPLE 3

Parenteral injection formulation

| | Amount |
|---|---|
| Active Ingredient | 10 to 300 mg |
| Citric Acid Monohydrate | 0.75 mg |
| Sodium Phosphate | 4.5 mg |
| Sodium Chloride | 9.0 mg |
| Water for injection | to 10 ml |

The sodium phosphate, citric acid monohydrate and sodium chloride are dissolved in a portion of the water. The active ingredient is dissolved or suspended in the solution and made up to volume.

### EXAMPLE 4

Double-Blind, Placebo-Controlled Study to Determine the Effect of a Substance P Receptor Antagonist on Patients Suffering Hormonal Variation

Approximately twenty patients diagnosed as suffering from hormonal variation receive either the substance P receptor antagonist 2-(R)-(1-(R)-(3,5-bis(trifluoromethyl)-phenyl)-ethoxy)-3-(S)-(4-fluoro-phenyl)-4-(3-(5-oxo-1H,4H-1,2,4-triazolo)methyl-morpholine (30 mg/day) or a placebo. Each subject participates in 6 randomized test periods; in 3 of the test periods, each is given the substance P receptor antagonist and in the other 3 test periods, is given a placebo. Efficacy of the test compound is assessed by reference to immunological profile, rating scales, checklists and diminishment of the attendant diseased state. The results of the foregoing study would indicate that the administration of a substance P receptor antagonist would be expected to have a positive effect with respect to placebo in the treatment or prevention of hot flashes following drug treatment.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims that follow and that such claims be interpreted as broadly as is reasonable.

1-24 (canceled)

25. A method for the treatment or prevention of vasomotor symptoms associated with hormonal variation in a patient in need thereof comprising administering an effective amount of a tachykinin receptor antagonist.

26. A method for the treatment or prevention of hot flashes in a patient in need thereof comprising administering an effective amount of a tachykinin receptor antagonist.

27. A method for the treatment or prevention of menopausal hot flashes in a female patient in need thereof comprising administering an effective amount of a tachykinin receptor antagonist.

28. The method of claim 25 wherein the tachykinin receptor antagonist is a neurokinin-1 receptor antagonist.

29. The method of claim 28 wherein the neurokinin-1 receptor antagonist is an orally active neurokinin-1 receptor antagonist.

30. The method of claim 29 wherein the neurokinin-1 receptor antagonist possesses a long duration of action.

31. The method of claim 25 wherein the patient is a human.

32. The method of claim 31 wherein the patient is a woman.

33. The method of claim 32 wherein the patient is an elderly woman.

34. The method of claim 32 wherein the patient is a post-menopausal woman.

35. The method of claim 25 wherein the hormonal variation is naturally induced, drug-induced or surgically induced.

36. The method of claim 25 wherein the hormonal variation is induced by menopause, premature menopause, anti-estrogen therapy, anti-androgen therapy, hysterectomy, oopherectomy or orchiectomy.

37. The method of claim 25 wherein the tachykinin receptor antagonist is employed in conjunction with an agent selected from the group consisting of: estrogen receptor modulators, androgen receptor modulators, and peptide hormones.
